(19) United States
(12) Patent Application Publication
(10) Pub. No.: US 2019/0201642 A1
(43) Pub. Date: Jul. 4, 2019

(54) HYDROGEN GAS ASPIRATOR

(71) Applicant: Takashi TAKEHARA, Osaka (JP)

(72) Inventor: Takashi TAKEHARA, Osaka (JP)

(21) Appl. No.: 16/331,715

(22) PCT Filed: Sep. 6, 2017
(86) PCT No.: PCT/JP2017/032172

§ 371 Date: Mar. 8, 2019

(30) Foreign Application Priority Data
Sep. 11, 2016 (JP) 2016-177192

(51) Int. Cl. A61M 15/02; A61M 15/00; A61M 15/06; C01B 3/08; A61K 33/00

(52) U.S. Cl. CPC A61M 15/02; A61M 15/0021; A61M 2202/02; C01B 3/08; A61K 33/00; A61M 15/06

(57) ABSTRACT

Provided is a hydrogen gas aspirator which can be carried by a user and freely carried by the user, and which can be supplied with hydrogen gas by adjusting concentrations of the aspirator. The hydrogen gas aspirator for generating and aspirating hydrogen gas includes: a first hydrogen gas aspirator body having at least a hollow interior; a second hydrogen gas aspirator body having an aspirator hollow layer and an aspirator hollow layer connected to each other by a low pressure check valve; and a variable pressure control valve capable of adjusting and passing hydrogen gas concentrations between the first and second hydrogen gas aspirator bodies, wherein substances that hydrogen generate and react by contacting water and water for reaction can be placed on the aspirator hollow layer inside the second hydrogen gas aspirator body.

### TECHNICAL FIELD

The present disclosure relates to a portable hydrogen gas aspirator capable of easily supplying a predetermined quantity of hydrogen gas.

### THE BACKGROUND OF THE INVENTION

In recent years, there have been provided hydrogen effectiveness in various animal disease experiments such as a neurodegenerative and an acute lung injury, and human clinical tests in metabolic syndrome and diabetes, and various researches have been actively carried out in a medical application. Since hydrogen removes only the bad reactive oxides (=hydroxyl radicals), which are responsible for promoting aging and causing various diseases such as arteriosclerosis and carcinoma, and does not adversely affect tissues and cells of the body, there are a wide range of methods for taking them into the body, including intravenous administration, oral administration of aqueous solution, and inhalation of gas.

In order to prevent the aging in various states such as during exercise, during eating and drinking, during smoking, during staying under ultraviolet light and contaminated environment, and during being highly stressed due to lack of sleep and long-hour working, in which the active oxygen particularly tends to be generated in the body, and promote beauty and health, it is suggested to take hydrogen in the body.

Conventionally, there have been generally two methods for generating hydrogen, and as a first method, there has been used a hydrogen generator of a desktop type or the like capable of generating hydrogen gases by placing water in a electrolysis tank on which an electrolysis plate having an ion exchange membrane, a pair of electrode plates in close contact with both surfaces of the ion exchange membrane, and a fixing portion in which the pair of electrode plates in close contact with both surfaces of the ion exchange membrane is mounted, and energizing the electrolysis plate. In this hydrogen generation device, since the user can use while optionally moving, usability is improved in comparison with the hydrogen generation device which can be used only in a stationary state.

However, in the above-mentioned conventional hydrogen generating device of the tabletop type or the like, it is required to secure a power source from an outlet in order to use a miniaturized device, and there is a limitation in a moving range in order to use the device as a hydrogen gas aspirator to be aspirated into a body by a user. In addition, when a battery with a built-in power source is used, it is essential to secure a space in which a battery is built in and to shield water between electrolysis tank ▪ battery, which limits downsizing and inevitably increases costs, which makes it difficult for many users to easily obtain the space or use the space at a destination.

As a second hydrogen generation method, a method using a hydrogen generation chemical reaction such as a chemical reaction between magnesium particles and water is also known. In this method, for example, a hydrogen generating case (see FIG. 4) in which magnesium particles and water are chemically reacted is provided, as in U.S. Pat. No. 2. In this instance of the case for hydrogen generation, there is employed a structure which performs the hydrogen generation reaction in an inner portion and discharges only the hydrogen to an outside through a hydrogen permeable film disposed in the case, thereby being compact and portable.

However, in the case of the hydrogen generating case of the above-mentioned document 2, it is used only for the purpose of charging it into a container such as a PET bottle, changing the moisture inside the container to hydrogen water, and making it drinking water, and it cannot be used as a hydrogen gas aspirator as it is. Further, even if the structure of the hydrogen generation case in the patent literature 2 is design changed for the hydrogen gas aspirator into a structure which can directly suck, a step of opening the case for pouring water when using and causing reaction between the magnesium particles in the inner portion with water is essential. Specifically, the user is allowed to secure water for water injection, labor is required for case opening work and water injection work at the time of water injection, and water may be wet at the time of water injection work.

In addition, when the above-mentioned conventional hydrogen generating case is used as a suction tool, a water injection tool, a cover for water injection, a valve of an aspirator main body, and the like are required in addition to the hydrogen generating device, and the number of components is increased, the shape of the aspirator main body is complicated, and the volume is increased, so that there are many problems in terms of products and transportation costs. Furthermore, since water is injected from one end of the aspirator main body, it takes a predetermined period of time before the water contacts metals such as magnesium particles in the container, and it is difficult to sufficiently satisfy the user's desire for rapid hydrogen suction. As a result, it has been impossible to provide a small and inexpensive disposable product which can be carried until the time of use and can be easily used in a desired place to suck hydrogen.

### ART DISCUSSED ABOVE

### Patented Literature

JP-A-2012-289290

Japanese Patent Application Laid-Open No. 2004-41949

[Patent Document 3] International Publication Gazette WO2015011046

### DISCLOSURE OF INVENTION

### Problems to be Solved by the Invention

The present invention has been made in view of the above circumstances, and an object of the present invention is to provide an inexpensive hydrogen gas aspirator which can be carried freely by a user and can easily and hygienically suction hydrogen gas when used.

### Means for Solving the Problem

In order to solve the above-mentioned problems, the hydrogen gas aspirator of the present invention is provided,

a longitudinally extending generally cylindrical aspirator main body having an opening at least at one end,

a mouthpiece member having a through-hole connectable or detachable to the open side of the aspirator main body and fluidly connected to said opening when connected,

a hydrogen generator that is generally rod-shaped and is longitudinally disposed within the aspirator main body,

The hydrogen generator of the hydrogen aspirator,

A longitudinally extending, longitudinally extending ampoule member containing a water-based aqueous solution, which can be cleaved by lateral manual force,

A reaction comprising metallic materials in the form of granules or powders which react ion means with water to generate hydrogen gases,

A generally cylindrical container body extending longitudinally, comprising: a material having at least lateral flexibility; and a reaction means and the ampule member arranged longitudinally therein, the hydrogen gas passing from the interior to the exterior and comprising a breathable impermeable material that does not penetrate the aqueous solution from the interior to the exterior.

In this hydrogen, when the gas suction tool is used, the hydrogen generator is inserted into the aspirator main body and positioned while the inner ampule member is cut by folding the container body of the hydrogen generator by lateral manual force.

In the present hydrogen gas aspirator, when the gas aspirator used, first, if the gas aspirator is folded by lateral manual force in the vicinity of the center of an elongated hydrogen generator having a generally cylindrical shape or the like, only the ampule member having low flexibility inside is cut without breaking or tearing the container body, and all of the aqueous solution enclosed in the ampule portion is discharged into the container body at once. As a result, aqueous solution and metallic materials chemically react with each other in the container body to generate hydrogen gases. The hydrogen gases are discharged through the breathable impermeable materials into the interior of the aspirator main body and into the user's mouth through the mouthpiece member.

According to this hydrogen gas aspirator, it is possible to manage without performing any hydrogen generation reaction at the time of inventory or carrying, but it is possible to suck a predetermined quantity of hydrogen gas into the mouth until the reaction is completed by inserting the hydrogen generator into the aspirator main body by simply folding it by the user's manual force at the time of use, and it is highly convenient without water injection operation. In addition, the hydrogen generator is hygienic without leakage of liquid to the user because it is covered by the container body even if the ampule member containing the aqueous solution is broken by folding it by manual force. In addition, the hydrogen generator is convenient because it can be disposable for each suction.

In addition, it is preferable that the metal material has a structure in which a metal is fixed to a surface of a granular material, a powder material, a non-reactant material, or a combination thereof.

In the generation of hydrogen, it is possible to adjust the quantity per hour, the duration and the reaction temperature of the desired hydrogen generation, and it is possible to obtain hydrogen water of the desired hydrogen concentration and the reaction temperature. Especially, it is easy to provide a product having a predetermined suction time or the like.

The ampule member may be disposed closer to the mouthpiece member than the reaction means.

Since the mouthpiece member usually has the suction port portion facing upward in many cases, if the suction tool is installed in this order, it is possible to avoid stagnation of the generation of hydrogen gases due to aqueous solution always being stored in the metallic material.

It is preferable that one end of the container body has an opening, and the opening is closed by a lid member having the breathable impermeable material.

Preferably, the container body is formed of a transparent or translucent member. This is because hydrogen aspiration can be realized by visually recognizing the hydrogen generation by the user.

The reaction means may be a generally rod-shaped cartridge having a nonwoven fabric containing the metal materials. In this hydrogen gas aspirator, the aspirator main body is usually repeatedly used, the hydrogen generator is disposable, and the cartridge-type device is easier to install and easier to manage and carry as a disposable product.

Further, a choke valve which is opened only when a negative pressure acts on the opening side may be disposed inside the vicinity of the opening of the aspirator main body, and an outside air intake port may be provided on the upstream side of the choke valve.

In this configuration, when the user inhales and applies a negative pressure to the opening of the aspirator main body, the choke valve is opened and hydrogen gases can be taken into the mouth, but on the contrary, external air does not flow into the aspirator main body from the suction port. In addition, when the negative pressure is applied at the time of suction, the outside air flows in from the outside air intake port, so that the hydrogen gases can be sucked in easily.

The breathable impermeable material is preferably a thermoplastic resin, and is, for example, at least one selected from the group consisting of (A) polyvinylidene chloride, (B) polyvinyl chloride, and (C) polyacrylonitrile. The metallic materials contain, for example, Mg, Al, and Ca as main components.

In addition, in the above-described hydrogen gas aspirator, the hydrogen generation device which is replaced every use is also separately managed. Specifically, for hydrogen generators,

A longitudinally extending, longitudinally extending ampule member containing a water-based aqueous solution, which can be cleaved by lateral manual force,

A reaction means comprising metallic materials in the form of granules or powders which react with water to generate hydrogen gases,

Comprising: a longitudinally extending generally cylindrical container body comprising at least a laterally flexible material, internally longitudinally disposing the reaction means and the ampule member in sequence, and comprising a breathable impermeable material that allows the hydrogen gases to pass from the interior to the exterior and prevents the aqueous solution from penetrating from the interior to the exterior;

In use, the container body of the hydrogen generator can be folded by lateral manual force to break the inner ampule member.

### Effect of the Invention

According to the present invention, it is possible to provide an inexpensive hydrogen gas aspirator which the user can bring and freely carry with and can easily and sanitarily suck hydrogen when using. According to this hydrogen gas aspirator, the user can suck hydrogen gas into a mouth until the end of the reaction only by bending the hydrogen generator with the user's hand power when using, a water pouring work is not necessary and the liquid leakage to the user does not occur. Further, the hydrogen generator is convenient for carriage and inventory control and can be a simple and disposable product.

### BRIEF DESCRIPTION OF THE DRAWINGS

FIG. 1 is a schematic cross-sectional view of one embodiment of the inventive hydrogen generator.

FIGS. 2A-2B are schematic diagrams of the inventive hydrogen generator in which hydrogen is generated by folding (FIG. 2A) from the unused state of FIG. 1 to the folded state of the outer coat member, and FIG. 2B from the folded state to the original state after folding.

FIGS. 3A-3B are schematic cross-sectional views illustrating an outer coat member having a convex shape portion protruding inwardly around the inner wall near the center of the center, and a part of the ampule member having a thin wall having a thickness smaller than that of the other part.

FIG. 4 is an axial cross-sectional view showing a first configuration of the hydrogen gas aspirator according to the present embodiment.

FIG. 5 is a schematic cross-sectional view of metallic particles composed of a generally spherical non-reaction portion and metallic particle layers coated and fixed on its surfaces.

FIG. 6 is an axial cross-sectional view of the present hydrogen gas aspirator.

FIG. 7A shows a perspective view of the hydrogen gas aspirator of FIG. 6, and FIG. 7B shows a perspective view of the ampule member.

### DETAILED DESCRIPTION OF THE INVENTION

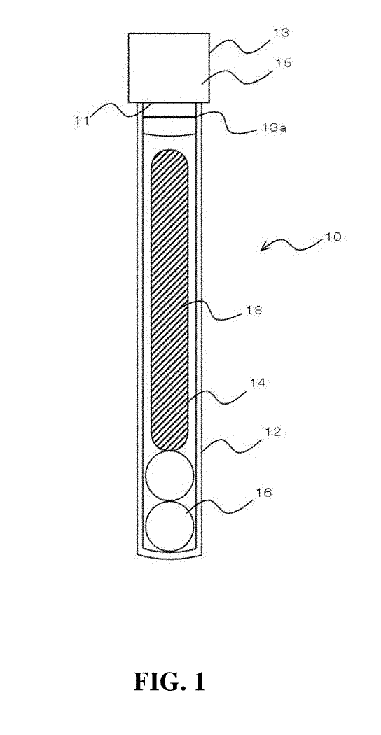

An exemplary embodiment of the hydrogen gas aspirator of the present invention will be described in detail below with respect to FIGS. 1 to 7B, but it is needless to say that the present invention is not limited to the one shown in the drawings. Further, each of the drawings is provided for conceptually describing the present invention and may be shown in an exaggerated manner or a simplified manner in dimensions, ratios or numbers as occasion demand for easy understanding. Further, in the following description, the same reference numerals are attached to the same or corresponding portions, and an overlapping description may be omitted.

First, the disposable and replaceable hydrogen generator 10 inserted into and installed in the main body of the hydrogen gas aspirator will be described. FIG. 1 is a schematic cross-sectional view of one embodiment of the inventive hydrogen generator 10. As shown in FIG. 1, in the hydrogen generator 10, a ampule member 14 and a metal member (metallic materials) 16 are enclosed in a outer coat member 12, and the inside of a outer coat member 12 is enclosed. A hydrogen passing member 13 is arranged above the outer coat member 12 (an upper side to the page space), and an aqueous solution 18 is sealed within the ampule member 14.

Subsequently, a description will be in detail given below of each of constituent elements.

The outer coat member 12 has a generally cylindrical shape extending longitudinally and having an open upper side (a mouthpiece member side of a hydrogen gas aspirator, which will be described later). The outer coat member 12 is made of a highly flexible material that can be bent or bent by hand while substantially preventing gases such as the metallic member 16, the aqueous solution 18, and the hydrogen from passing therethrough, and is generally made of a resin material such as rubber. The ampule member 14 and the metal member 16 are encapsulated by a hydrogen passing member 13 (lid member 15 to be described below) that connects the outer coat member 12 to the opening of the outer coat member 12.

A generally cylindrical cork-shaped lid member 15 is press-fitted into the opening 11 at the upper end of the outer coat member 12, and a thin film material 13 a is provided on the lower surface of the lid member 15. The film raw material 13a is constructed by an air-permeable and water-impermeable material which does not allow passage of the metal and the aqueous solution and allows passage of the gas. Further, the lid member 15 is constructed by a raw material which allows passage of the gas, and hydrogen generated within the outer coat member 12 is discharged outward via the hydrogen passing member 13 which is constructed by the film raw material 13a and the lid member 15.

The lid member 15 of the breathable impermeable material 13 is a nonwoven fabric or the like, and the film material 13 a is, for example, a thermoplastic resin such as polyvinylidene chloride, polyvinyl chloride, or polyacrylonitrile.

The ampule member 14 is formed into an approximately tubular shape (a rod-like member) extending in the longitudinal direction which forms a closed space sealing the water-based queous solution. The ampule member 14 has an outer wall made by a raw material such as glass or plastic which is harder than the outer coat member 12 and tends to be ruptured, in the same manner as a general-purpose ampule. Actually, it can be easily ruptured when a hand power is applied thereto in an approximately perpendicular direction with respect to the longitudinal direction without being bent.

Further, since the ampule member 14 has a higher rigidity than the outer coat member 12, the ampule member 14 is ruptured before the outer coat member 12 is ruptured or torn by applying a pressure to the outer coat member 12 in the perpendicular direction with respect to the longitudinal direction, so that the aqueous solution 18 sealed within the ampule member 14 flows out into the outer coat member 12 and is accumulated.

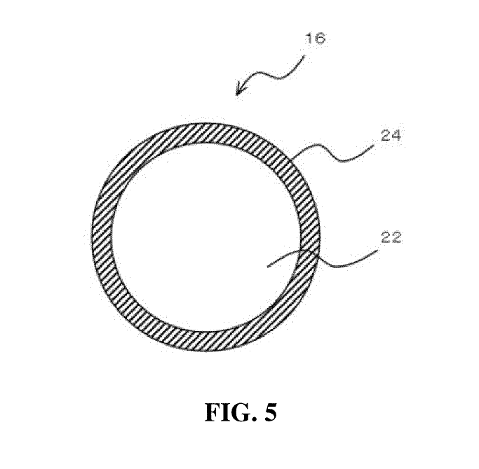

FIG. 1 schematically shows 12 example of a gr12ular shape as the metal member 16 sealed within the outer coat member 16. Specifically, each metallic member 16 is comprised of a generally spherical non-reaction portion 22 and metallic particle layers 24 clad thereon, as shown in FIG. 5. The metallic particle layers 24 react with water to generate hydrogen. For example, the metal is magnesium, aluminum, calcium, potassium, sodium, zinc or iron. The non-reaction portion 22 is preferably made of a raw material which does not substantially generate any chemical reaction by contacting with the aqueous solution, for example, plastic or a ceramic ball. The metallic particle 24 is fixed to the surface of the non-reaction portion 22, for example, by baking. As a result, the surface area of the metal member 16 contacting the aqueous solution can always be secured, and the aggregation of the metal particles does not occur at the time of reaction with the aqueous solution 18 as compared with the case of placing the metal member 16 as it is in a powder state, and a stable hydrogen generation reaction can be secured.

In order to avoid aggregation of metallic particle, cartridges in which metallic particle layers 24 are applied to a non-reaction portion 22 such as a nonwoven fabric or the like and modified are also conceivable. When the diameter of the outer coat member 12 is large to some extent, or when hydrogen is generated and reacted quickly, the powdery metallic particle layer 24 may be placed on the layer as it is.

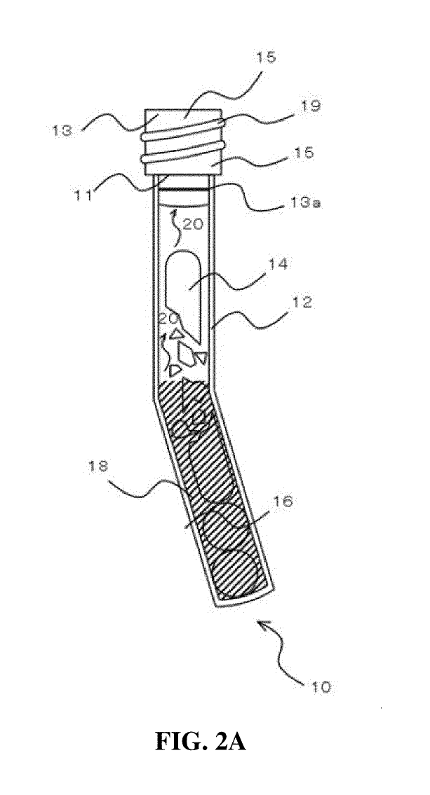

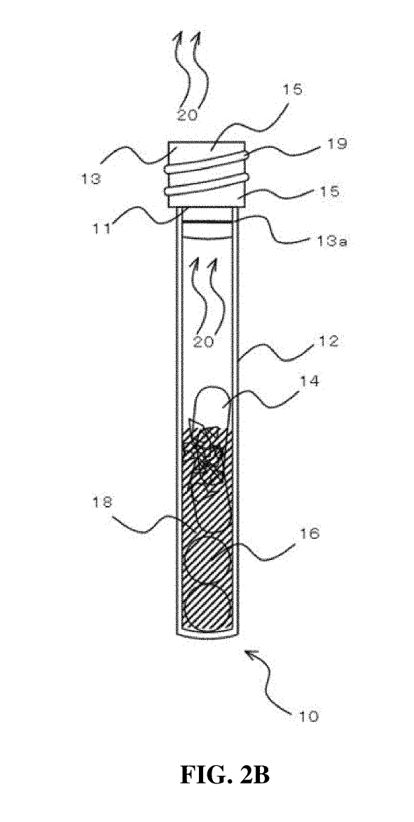

Next, a description will be given of a method of using the present hydrogen generator 10 with reference to FIGS. 2A and 2B. FIGS. 2A and 2B are schematic diagrams of the inventive hydrogen generator 10 for generating hydrogen by folding, wherein FIG. 2A shows a state in which the outer coat member 12 is folded from a state in which it is not in use in FIG. 1, and FIG. 2B shows a state in which it is returned to an original state after folding. When pressure is applied in a direction perpendicular to the longitudinal direction of the hydrogen generator 10, the outer coat member 12 is bent and the ampule portion 14 is cut as shown in FIG. 2A. In connection therewith, the aqueous solution 18 within the ampule member 14 flows out into the outer coat member 12, and comes into contact with the metal member 16. Subsequently, as shown in FIG. 2B, chemical reactions between water and metal occur, as a result of which hydrogen 20 is generated, and only the hydrogen 20 flows out of the outer coat member 12 through the film material 13 an of the upper cover member 15.

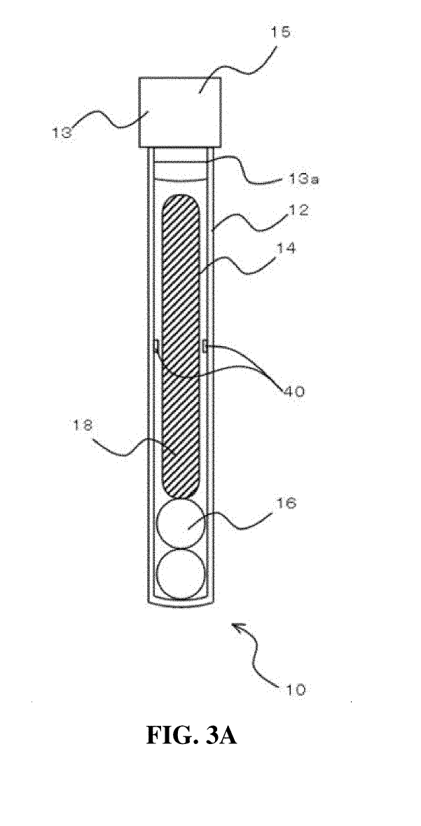

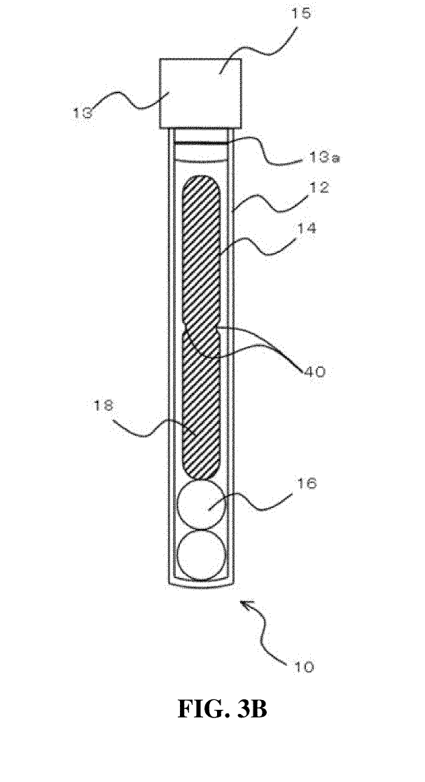

The outer coat member 12 has a reduced thickness or diameter in the vicinity of the longitudinal center. For example, in the example of FIG. 3A, a convex shape portion 40 protruding inward around the inner wall near the center of the outer coat member 12 is provided. With this convex shape portion 40, when a lateral load is applied to the hydrogen generator 10, not only the convex shape portion 40 is stress-concentrated and the part thereof is easily bent, but also the ampoule member can be fractured more easily by contacting and pressurizing the vicinity of the longitudinal center of the ampule member 14.

In the meanwhile, in an example of FIG. 3B, a part of the ampule member 14 is provided with a thin portion 41 which has a smaller thickness than in the other portion. Since the thin portion 41 tends to be ruptured in comparison with the other portions, the ampule member 14 is ruptured easily with the smaller transverse load (pressing force). Further, the thin portion 41 is provided in the vicinity of the center of the ampule member 14 in the longitudinal direction, and the aqueous solution 18 rapidly flows out into the outer coat member 12 in comparison with the case that only one end is ruptured. Further, in the case that the convex shape portion 40 of the outer coat member 12 mentioned above is arranged at the approximately same position as the thin portion 41 in the longitudinal direction, the ampule member 14 is more easily ruptured.

As the metallic member 16, for example, magnesium, aluminum, calcium, or a hydrogen of magnesium, aluminum, calcium, or the like is exemplified as the main composition which reacts with water to release hydrogen gas.

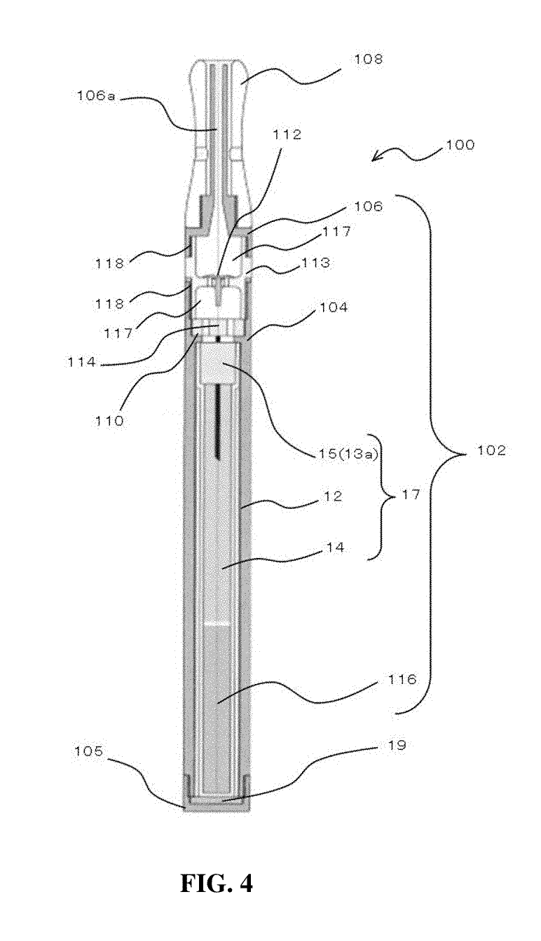

Having illustrated and described the hydrogen generator 10 of the type enclosing the ampule member 14, two exemplary configurations of the inventive hydrogen gas aspirator to which the hydrogen generator 10 can be replaceably mounted are now described with respect to FIGS. 4 and 6-7B. FIG. 4 is an axial cross-sectional view showing a first configuration of the hydrogen gas aspirator according to the embodiment of the present disclosure. In this hydrogen gas aspirator 100, the mouthpiece member 108, the connection portion 106, and the aspirator main body 102 are arranged in order from the right side of the paper plane (the user's mouth side). A hydrogen generator 10 is disposed axially along the aspirator main body 102. It can also be said that the coupling portion 106 is formed as a part of the aspirator main body 102.

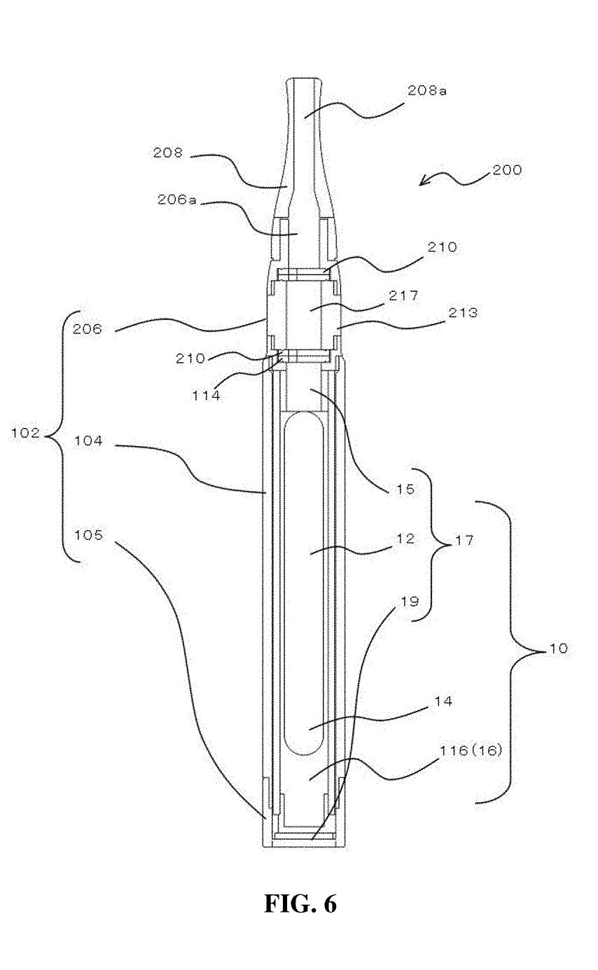

As described above with reference to FIG. 1 and the like, the hydrogen generator 10 includes a ampule member 14 and metallic materials inside a container body 17. In the embodiment shown in FIG. 6, rod-shaped cartridges 116 containing metallic particle in a nonwoven fabric are arranged as metallic materials. In FIG. 6, the bottom portion of the container body 17 is closed at the bottom end of the outer coat member 12 by a cap-shaped closing member 19. An end portion of the outer coat member 12 on the right side in the drawing is closed by a cover member 15. The lid member 15 is hydrogen passing member, but in the case of a hydrogen passing member such as a nonwoven fabric, the film material may be disposed at an end which does not allow water to pass through, as in the example of FIG. 1, and the film packing 110 which is layered through a film material of a breathable impermeable material may be disposed.

A coupling portion 106 is coupled to an end portion of the aspirator main body 102. The opening at the end of the aspirator main body 102 and the connection 106 provide a gap 117 in which the regulating valve 112 is disposed. In addition, an O-ring 118 is mounted around the shaft at the connection portion between the connection portion 106 and the aspirator main body 102 in view of the risk of leakage of water vapor from the hydrogen generator 10 for a long time or during repeated use. The regulating valve 112 is a valve that is opened when a negative pressure acts on the right side of the drawing, and is closed when the negative pressure disappears. The regulating valve 112 allows the user to draw in the hydrogen gas-containing air only when the user desires to draw in the hydrogen gas. A window 113 is provided on the outer wall of the connecting portion 106 to allow the operation of the regulating valve 112 to be seen in order to make it easier to understand how the hydrogen gases are sucked in.

A hollow cylindrical member 106 a extends from the distal end of the connecting portion 106, and a mouthpiece member 108 is nested over the cylindrical member 106 a. The mouthpiece member 108 is press-fitted to the cylindrical member 106 a by manual force.

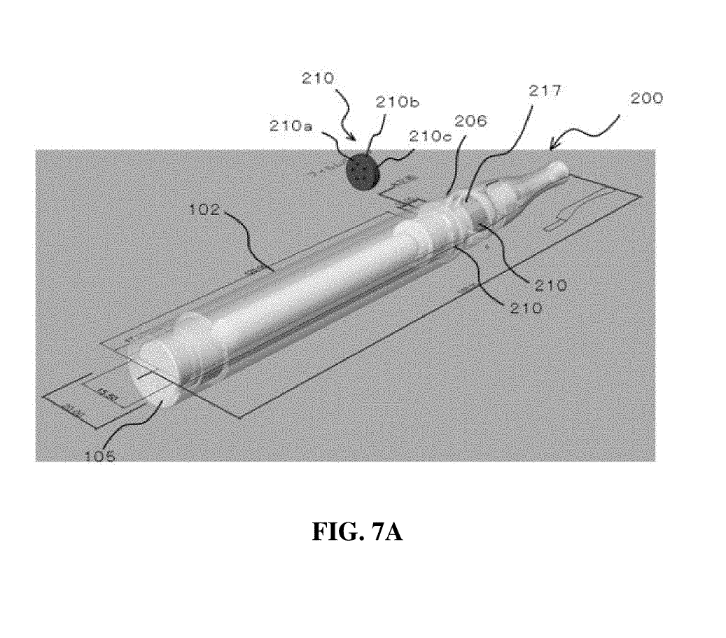

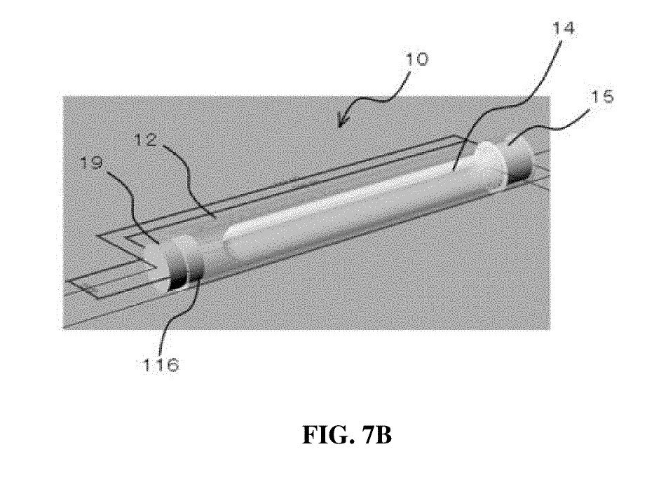

Referring now to FIGS. 6-7B, the configuration of a second hydrogen gas aspirator 200 is illustrated. FIG. 6 shows an axial cross-sectional of the hydrogen gas aspirator 200, and FIGS. 7A and 7B show a perspective view thereof in part (FIG. 7A) and a perspective view of the ampule member in part (FIG. 7B). In the configuration examples of FIGS. 6-7B, substantially the same members as those of the configuration examples of FIGS. 1 to 5 are denoted by the same reference numerals, and different reference numerals 206 and 208 are denoted for the connecting portion and the mouthpiece member which are greatly deformed from those of the example of FIG. 4.

First, as described above, the hydrogen generator 10 in the hydrogen gas aspirator 200 of FIGS. 6 to 7B has the ampule member 14 and the cartridges 116 (or 16) enclosed inside the container body 17, and both ends of the outer coat member 12 of the container body 17 are closed by the closing member 19 and the cover member 15. The cover member 15 is made of hydrogen passing member, and a pair of film packings 210, which will be described later, are disposed on the breathable impermeable material which allows only hydrogen gas to pass therethrough.

A connecting portion 206, which is largely different from that of FIG. 4, is connected to the end portion of the aspirator main body 102. In the case of the coupling portion 206, a gap 217 is provided by the opening of the end portion of the aspirator main body 102 and the coupling portion 206, and a film packing 210 for closing the opening is attached to each of both ends. Film packing 210 is formed by sandwiching film 210b with disc members 210a, 210c shaped to close both end openings of connections 206 as shown in FIGS. 7A-7B removed from hydrogen gas aspirator 200. The disk members 210 a and 210 c are formed of a non-reactive member such as a resin material similar to that of the O-ring 118, and are each provided with a plurality of through holes penetrating in the thickness direction (axial direction). The film 210 b is a membrane-like member of a breathable impermeable material formed of a thermoplastic resin that passes hydrogen gases such as polyvinylidene chloride, polyvinyl chloride, polyacrylonitrile, or the like and does not allow water to pass therethrough.

Therefore, in use, the hydrogen gas or water vapor discharged through the cover member 15 at the upper end of the hydrogen generator 10 passes through the through hole of the disk member 210 a, the gas such as hydrogen gas passes through the film 210 b, and flows from the through hole of the disk member 210 a into the gap 217 in the connecting portion 206. The space 217 is filled with the aroma solution, and the hydrogen gas passes through the solution, and the perfumed hydrogen gas reaches the film packing 210 disposed at the opening of the connection portion 206 on the side of the mouthpiece member 208.

Thereafter, the gas passes through the through-holes of the disk member 210 a, and the perfumed hydrogen gas is discharged from the through-holes of the disk member 210 a through the films 210 b. The released perfumed hydrogen gases pass through the through-hole 208 a extending axially in the mouthpiece member 208 from the opening 206 a at the end of the connecting portion 206 and are discharged into the outside user's mouth. It should be noted that the mouthpiece member 208 is put on the distal end of the connecting portion 206 by manual force in a nested manner. This is the same as the example of FIG. 4.

While embodiments of the hydrogen gas aspirator of the present invention have been illustrated and described above, those skilled in the art will appreciate that the present invention is not limited thereto, and that other modifications and improvements can be obtained without departing from the spirit and teachings of the claims and the specification.

### INDUSTRIAL APPLICABILITY

According to the inventive hydrogen gas aspirator, the hydrogen gas can be sucked into the mouth until the completion of the reaction by simply folding the hydrogen generator by the user's manual force at the time of use, and the hydrogen generator can be made a disposable product which is easy to carry and manage the inventory of the product without the water injection operation and without the leakage of the liquid to the user.

### EXPLANATION OF REFERENCE NUMERALS

10 Hydrogen generator
11 Opening
12 Outer coat member
13 Hydrogen passing member
13a Film material (breathable impermeable material)
14 Ampule member
15 Lidding member
16 Metal materials
17 Container body
18 Aqueous solution
19 Closure member
20 Hydrogen
22 Non-reaction portion
24 Metallic particle layers
40 Convex shape portion
41 Thinned portion
100 200 Hydrogen gas aspirator
102 Aspirator main body
106 206 Interconnection
108 208 mouthpiece member
110 210 Film packing
112 Regulating valve
113 213 Window
116 Cartridge
117 217 Gaps
118 O-ring

1. A hydrogen gas aspirator comprising:
a longitudinally extending generally cylindrical aspirator main body having an opening at least at one end;
a mouthpiece member having a through-hole connectable or detachable to the open side of the aspirator main body and fluidly connected to the opening when connected;
a hydrogen generator that is generally rod-shaped and is longitudinally disposed within the aspirator main body;
and a breathable impermeable means for allowing the hydrogen gases to pass from the inside to the outside and for guiding the gases from the inside to the mouthpiece member without penetrating aqueous solution from the inside to the outside,
wherein the hydrogen generator comprises a longitudinally extending generally cylindrical ampule member containing a water-based aqueous solution, which can be cleaved by lateral manual force,
a reaction means comprising metallic material that react with water to generate hydrogen gases and,
a longitudinally extending generally cylindrical container body comprising at least a laterally flexible material, a longitudinally disposed longitudinally of the reaction means and the ampule member,
in use, the hydrogen gas aspirator wherein the hydrogen generator is inserted and positioned within the aspirator main body with the interior ampule member cut off by folding the container body of the hydrogen generator with lateral manual force.

2. The hydrogen gas aspirator as claimed in claim 1, wherein said metallic materials are particulate, powdered, fixed to non-reactant surfaces, or combinations thereof.

3. The hydrogen gas aspirator according to claim 1, wherein the ampule member is disposed closer to the mouthpiece member than the reaction means.

4. The hydrogen gas aspirator of claim 1, wherein one end of the container body has an opening closed by a closure member having the breathable impermeable material.

5. The hydrogen gas aspirator according to claim 1, wherein the container body is formed of a transparent or translucent member.

6. The hydrogen gas aspirator according to claim 1, wherein the reactor is a generally rod-shaped cartridge having a nonwoven fabric containing the metallic material.

7. The hydrogen gas suction tool according to claim 1, wherein a regulating valve which is opened only when a negative pressure acts on the opening side is disposed inside the vicinity of the opening of the aspirator main body, and an outside air intake port is provided on the upstream side of the regulating valve.

8. The hydrogen gas aspirator according to claim 1, wherein the breathable impermeable materials are thermoplastics resin.

9. The hydrogen gas aspirator as claimed in claim 8, wherein said thermoplastic resin is at least one selected from the group consisting of (A) polyvinylidene chloride, (B) polyvinyl chloride, and (C) polyacrylonitrile.

10. The hydrogen gas aspirator according to claim 1, wherein the metallic material is composed mainly of Mg, Al, and Ca.

11. A hydrogen generator, comprising:
a longitudinally extending generally cylindrical ampule member containing a water-based aqueous solution, which can be cleaved by lateral manual force;
a reaction means comprising metallic materials that react with water to generate hydrogen gases;
in use, the interior ampule member cut off by folding the container body of the hydrogen generator with lateral manual force.
